(19) United States
(12) Patent Application Publication
(10) Pub. No.: US 2008/0139637 A1
(43) Pub. Date: Jun. 12, 2008

(54) Catalyzed Reaction for Forming Indole-Based Compounds and Their Application in Anticancer Agents

(75) Inventors: Ching-Fa Yao, Taipei City (TW); Yi-Ching Wang, Taipei City (TW)

Correspondence Address: WPAT, PC, 7225 BEVERLY ST., ANNANDALE, VA, 22003, US

(73) Assignee: NATIONAL TAIWAN NORMAL UNIVERSITY, Taipei City (TW)

(21) Appl. No.: 11/567,471

(22) Filed: Dec. 6, 2006

(51) Int. Cl. A61K 31/404; C07D 209/14; A61P 35/00; C07D 403/14

(52) U.S. Cl. 514/414; 548/490; 548/455

(57) ABSTRACT

The present invention discloses a method for forming indole-based compounds, wherein the method comprises a reaction of α,β-unsaturated ketone or aldehyde with indole or its derivative in the presence of at least one kind of Lewis acid. The Lewis acid comprises one of the following groups: metal halides, halogens, inorganic ammonia salts, organic sulfonate and sulfonic acid. Furthermore, this invention discloses 3-indole based compounds applied as anticancer agents.

### BACKGROUND OF THE INVENTION

1. Field of the Invention

The present invention is generally related to a method for forming indole-based compounds by a catalyzed reaction, and more particularly to a method for forming indole-based compounds catalyzed by a Lewis acid and their application in anticancer agents.

2. Description of the Prior Art

A “cancer cell” is so called because of the mutation of many cancer-related genes, such as tumor suppressor genes, proto-oncogenes, of the cell in a body to transform from a normal cell to a cancer cell. On the molecular level, unlike normal cells that are controlled by the death and growth mechanisms, cancer cells are not easy to be aging and died and have the capability of fast proliferation. From the past research findings, many growth-stimulating receptors and signal molecules (such as erb-B, HER-2, Ki-ras, c-myc) and anti-apoptosis molecules (such as Bcl-X_{L}) having the phenomena of overexpression and overactivation in cancer cells are called “oncogenes”. On the other hand, some important regulating molecules in the cell cycle, such as p53, p16, and pRb, may be deleted from genome library, or these molecules on the DNA promoter sequence sites are seriously methylation, or these molecules may have mutations. These result in abnormal functioning of these molecules. Thus, the cell cycle is carrying on without control to have cancer cells undergo continuous cell division and proliferation. These molecules are thus called “tumor suppressor genes”. Certainly, formation of cancer cells is not only simply because of several uncontrollable molecules but also involved with many known or unknown molecules. Due to the complexity of the cancer cells themselves, the difficulty in cancer treatment is increased.

In the prior research, the effect of chemotherapy is to cause necrosis of tumor cells. However, recent reports show that many anticancer agents cause physiological disorder of cells and thereby cause programmed cell death, i.e., apoptosis. The programmed cell death exists in almost all of the tissue cells. When cells are aging, damaged, or loss function, these useless cells are removed through suicidal behavior of the majority of cells, called “apoptosis” that is different from normal cellular necrosis. Because apoptosis does not cause inflammation in contrast to cellular necrosis and apoptotical cells are quickly decomposed by neighboring cells, cell death does not cause necrosis of neighboring cells and disorder of the immune system. In mammals, the earliest separated apoptotical molecule is the Bcl-2 gene. When the Bcl-2 gene is overexpression, apoptosis is suppressed. Some cancers rely on Bcl-2 and related genes to prevent cell death. Prognosis of prostate cancer and colorectal cancer is also related to the expression of Bcl-2. On the other hand, p53 gene has also been extensively studied. When DNA is damaged, p53 is overexpression to arrest cells in the G1/S stage. Until DNA is repaired, cells have normal cell cycle. However, when DNA is seriously damaged, p53 induces apoptosis. In many cancers, the defect of the p53 gene is detected. From current reports, the mutation of the p53 gene is the most likely happened in cancer cells.

Indole and its derivatives are well known as biologically active substances, for example having induction effect on apoptosis of cancer cells. Organic chemists have been paying attention to synthesize different kinds of indole compounds, including bis(indolyl)methanes, β-indolylnitro, β-indolylketone, and β-indolylalcohol compounds, etc. Until now, only two literatures have been reported about the preparation of the above compounds. For example, it has also been reported by Harrington and Keer that trisindolylcyclohexane was given in 6% yield under ultra high pressure condition (Harrington, P.; Keer, M. A. Can. J. Chem. 1998, 76, 1256) and Shi et al. have also reported that these compounds can be prepared by using metal triflate as catalyst (Shi, M.; Cui, S.-C.; Li, Q.-J. Tetrahedron 2004, 60, 6679). However, both methods have some disadvantages, such as harsh reaction condition (13 kbar), long reaction time (1-3 days), and the use of expensive metal catalysts. Therefore, research in forming indole-based compounds with low cost, low toxicity, unpolluted and easily operable process is still needed. Besides, new catalysis tacetic should be utilized to increase the production yield and speed. It is also an important development aspect in the industry.

### SUMMARY OF THE INVENTION

In light of the above background, the present invention provides a method for forming indole-based compounds by a catalyzed reaction and their application in anticancer agents, in order to meet the industrial requirements.

One object of the present invention is to use a Lewis acid as the catalyst to provide an easily-operable and stable process for forming indole-based compounds. The catalyst used in the invention has the advantages of high reaction efficiency, ease in handling reacted mixture, mild or proper reaction conditions, such as carrying out the reaction under room temperature, and producing single product with a medium to high yield rate. Therefore, the present invention does have the economic advantages for industrial applications.

Another object of the present invention is to provide a medical composition for cancer treatment. The medical composition comprises a 3-indole based compound, its enantiomers, diastereomers, and medical-allowable salts or any combination of the above, especially suitable for cancer cells with the mutation of the p53 tumor suppressor gene.

Accordingly, the present invention discloses a method for forming indole-based compounds, comprising a catalyzed reaction of α, β-unsaturated ketone and indole or its derivatives in the presence of at least one Lewis acid or a catalyzed reaction of α, β-unsaturated aldehyde and indole or its derivatives. The Lewis acid comprises one selected from the group consisting of the following: organic sulfonic acid (sulfonate), halogen, inorganic ammonium salt, and metal halide. On the other hand, the present invention also discloses the application of 3-indole based compounds on anticancer agents.

### BRIEF DESCRIPTION OF THE DRAWINGS

FIG. 1 shows the result of the survival rate of various cancer cells treated with 3-indole drug according to the average of three independent experiments. 3-indole could achieve an IC50 value at ˜10M in various cancer cells, whereas did not show apparent cyclotoxicity to the IMR-90 normal cells at this dose;

FIG. 2 shows the suppressing effect of 3-indole drug to in vivo animal test (A) The animals were implanted s.c. (Subcutaneous) with 5×10⁶ A549 lung cancer cells. After randomization, animals were treated i.p. (intraperitoneal) with 3-indole, Taxol at 0.2 mg/2 day (final dose 50 mg/Kg), or a vehicle mixture control. The traditional chemotherapy drug Taxol was included as a positive control. The solvent for 3-indole, DMSO is also used as a negative control. After 21 days of observation on the tumor size, animals were sacrificed and processed for evaluation of any possible (B) serum biochemical toxicities and (C) histopathologic damage;

FIG. 3 shows the cell cycle distribution diagram of the various lung cancer cells after 24 hrs treated or untreated with 3-indole drug according to the average of three independent experiments where G1 represents a cell with two sets of chromosomes (2N), G2/M represents a cell with four sets of chromosomes (4N), S is between G1 and G2/M, representing the cell population in DNA synthesis, sub-G1 represents chromosomes having DNA fragmentation in cells, possibly showing apoptosis, and besides solid arrows in the figure show the increase of the sub-G1 peak intensity and hollow arrows show the increase of the G2/M peak intensity;

FIG. 4 shows the result of DNA ladder assay by DNA electrophoresis according to the average of three independent experiments where various lung cancer cell lines treated with 30 μM of 3-indole drug for (A) 24 hrs and (B) 48 hrs; and

FIG. 5 is the result of Western Bolt analysis showing anti-apoptotic proteins Bcl-2 expression of the A549 and H1437 cancer cell after treated with 30 μM of 3-indole drug according to the average of three independent experiments. The GAPDH is used as an internal control for protein loading.

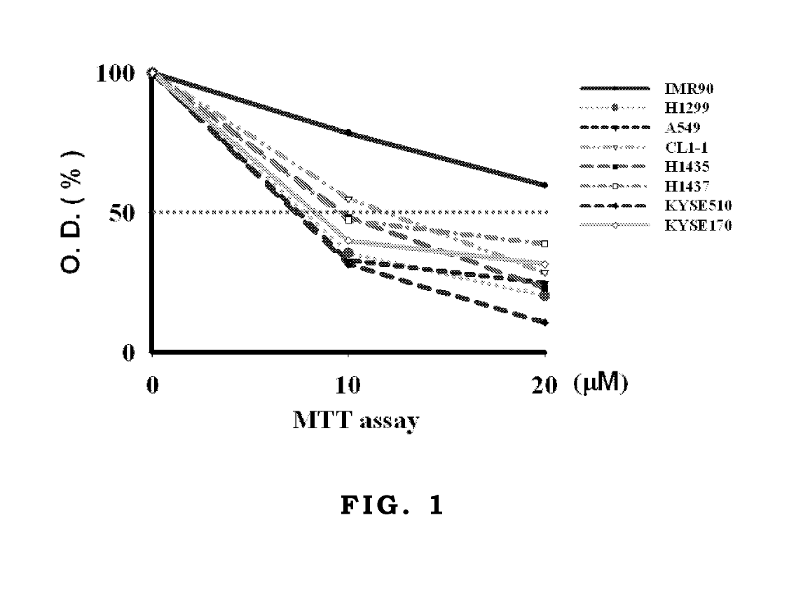

### DESCRIPTION OF THE PREFERRED EMBODIMENTS

What is probed into the invention is a method for forming indole-based compounds by a catalyzed reaction. Detail descriptions of the structure and elements will be provided in the following in order to make the invention thoroughly understood. Obviously, the application of the invention is not confined to specific details familiar to those who are skilled in the art. On the other hand, the common compositions and processes that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In a first embodiment of the present invention, a method for forming indole-based compounds is disclosed. The method comprises a room-temperature catalyzed reaction. The room temperature can be lower than or equal to 30° C. The catalyzed reaction is the reaction of α, β-unsaturated ketone and indole or its derivatives in the presence of at least one Lewis acid. The Lewis acid comprises one selected from the group consisting of the following: organic sulfonic acid (sulfonate), halogen, inorganic ammonium salt, and metal halide. The metal halide comprises one selected from the group consisting of the following: indium halide (InCl₃), cerium halide (CeCl₃), and aluminum halide (AlCl₃).

Moreover, the organic sulfonic acid comprises one selected from the group consisting of the following: alkyl sulfonic acid (sulfonate) (such as dodecyl sulfonic acid or dodecyl sulfonate), aryl sulfonic acid (sulfonate) (such as 4-methylbenzenesulfonic acid), alkyl aryl sulfonic acid (sulfonate) (such as dodecyl benzenesulfonic acid or dodecyl benzenesulfonate), sulfonated styrene-divinylbenzene copolymer, and Nafion. The sulfonated styrene-divinylbenzene copolymer is an acidic ion exchange resin and has been extensively utilized as a catalyst. Its commercial products comprise Amberlyst-15, Amberlyst XN-1005, Amberlyst XN-1010, Amberlyst XN-1011, Amberlyst XN-1008, Amberlite 200, and so forth.

In this embodiment, the α, β-unsaturated ketone comprises one selected from the following group:

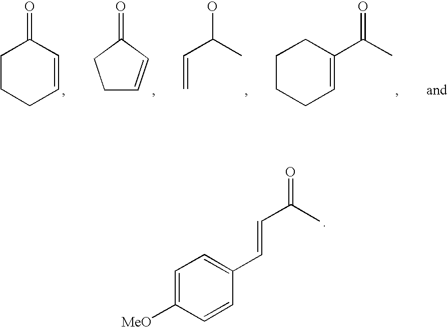

In addition, the general structure of said indole of its derivatives is as following:

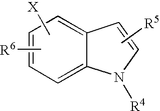

in which X is halogen and R⁴, R⁵, and R⁶ are independently selected from the group consisting of the following: hydrogen atom and linear-chained alkyl group (such as methyl or ethyl group).

In a second embodiment of the present invention, a method for forming indole-based compounds is disclosed. The method comprises a room-temperature catalyzed reaction. The room temperature can be lower than or equal to 30° C. The catalyzed reaction is the reaction of α, β-unsaturated aldehyde and indole or its derivatives in the presence of at least one Lewis acid. The selection of the Lewis acid, indole, or its derivatives are the same as those in the first embodiment. Besides, the α, β-unsaturated aldehyde comprises one selected from the following group:

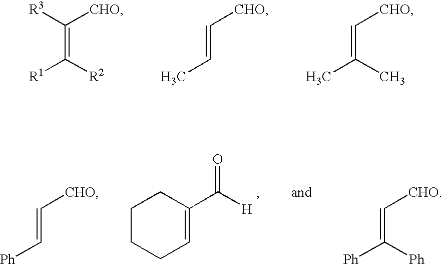

### EXAMPLE 1

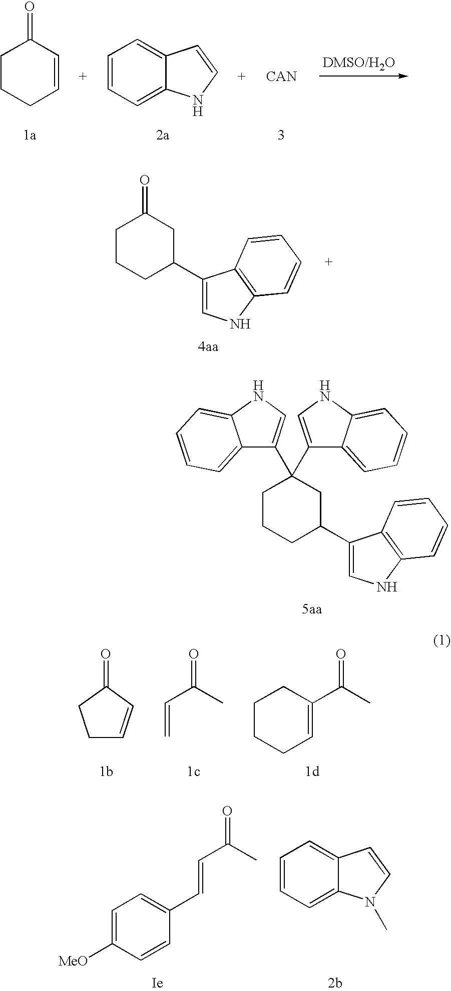

TABLE 1
Reaction of α, β-unsaturated ketone 1 and indole
2a in the presence of cerium ammonium nitrate

| | | 2 (4 | 3 | Time | | |
|---|---|---|---|---|---|---|
| Entry | 1 (1 equiv) | equiv) | (equiv) | (h) | 4^{a} (%) | 5^{a} (%) |
| 1 | 1a | 2a | — | 20 | 4aa (—) | 5aa (—) |
| 2 | 1a | 2a | 0.1 | 12 | 4aa (14) | 5aa (85) |
| 3 | 1a | 2a | 0.1 | 20 | 4aa (—) | 5aa (93) |
| 4 | 1a | 2a | 0.3 | 6 | 4aa (8) | 5aa (92) |
| 5 | 1a | 2a | 0.3 | 13 | 4aa (—) | 5aa (99) |
| 6 | 1a | 2a | 0.5 | 2 | 4aa (11) | 5aa (77) |
| 7 | 1a | 2a | 0.5 | 4 | 4aa (—) | 5aa (85) |
| 8 | 1b | 2a | 0.3 | 1 | 4ba (99) | 5ba (—) |
| 9 | 1c | 2a | 0.3 | ⅙ | 4ca (90) | 5ca (—) |
| 10 | 1d | 2a | 0.3 | 72 | 4da (62) | 5da (—) |
| 11 | 1e | 2a | 0.3 | 4 | 4ea (60) | 5ea (—) |

^{a}NMR yields.

To observe the catalytic effect of CAN, 1 equiv of 2-cyclohexen-1-one 1a was used to react with 4 equiv of indole 2a in the presence of different amounts of CAN in DMSO/H2O (5:1) solution at room temperature (Eq. 1 and Table 1). First, neither 1,4-addition product 4aa nor 1,4- and then 1,2-addition product 5aa was observed when the reaction was performed in the absence of any reagent for 20 h (entry 1). However, the reaction was improved dramatically and 14% of 4aa and 85% of 5aa were generated when the same reaction was carried out in the presence of 0.1 equiv of CAN for 12 h (entry 2). Surprisingly, only 93% of 5aa was obtained when the same reaction was carried out for 20 h (entry 3).

To improve the optimized yield of 5aa, the amount of CAN was increased to 0.3 equiv and, as expected, the yield of 4aa was decreased to 8% only but 5aa was increased to 92% for 6 h (entry 4). Fortunately, only 99% of the single product 5aa was generated when the same reaction was carried out for 13 h (entry 5). Although the increase of CAN to 0.5 equiv can accelerate the reaction dramatically, however, the yields of 4aa and 5aa were decreased to 11% and 77% for 2 h and only 85% of 5aa was observed for 4 h (entries 6 and 7). Possible explanation is that the starting material or product may be destroyed during reaction if the exothermic reactions occur too fast when excess amount of CAN was added. According to the above results we can conclude that CAN actually can catalyze the reaction efficiently to obtain high yields of 5aa and the best amount of CAN to be used in this reaction is 0.3 equiv. In addition to 1a, similar reactions were also conducted by using 1b-e, respectively, under similar conditions and the results were shown as entries of 8-11.

Based on the data of Table 1, we found different and interesting results were observed between 1a and 1b-e. Only 1a can undergo the 1,4-addition to yield 4aa first and then the intermediate 4aa can react with indole 2a to undergo 1,2-addition to obtain 5aa finally. However, 1b-e only can undergo 1,4-addition to yield medium to high yields of 4ba-ea, respectively. About the generation of the different products from 1a and 1b, we proposed that the torsional strain effect plays an important and major role to the product or intermediate during reaction. The addition of the first equivalent of 2a to 1a or to 1b forms 3-indolylcyclohexanone 4aa or 3-indolylcyclopentanone 4ba. When further reaction occurs, the conversion of the sp² carbon atom of the carbonyl functional group of 4aa to a sp³ of six-membered ring of 5aa leads to a completely staggered (chair) arrangement and reduces the torsional strain. On the contrary, the torsional strain is increased because of the increase in the number of eclipsing interaction which was generated from the further reaction product of 5ba.

### EXAMPLE 2

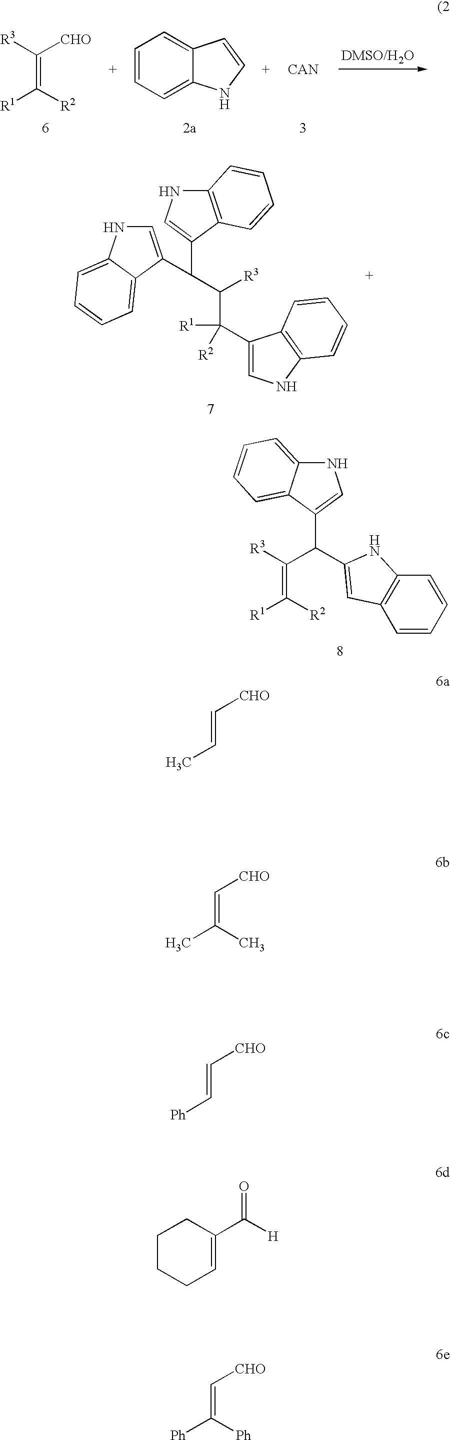

TABLE 2
CAN-catalyzed reaction of α, β-unsaturated aldehyde 6 with indole 2a

| Entry | 1 | 3 (equiv) | Time (h or min) | | 7^{a} (%) | 8^{a} (%) |
|---|---|---|---|---|---|---|
| 1 | 6a | 0.1 | 1 | h | 7aa (99) | 8aa (—) |
| 2 | 6b | 0.1 | 10 | min | 7ba (64) | 8ba (29) |
| 3 | 6c | 0.1 | 1 | h | 7ca (32) | 8ca (66) |
| 4 | 6d | 0.1 | 5 | min | 7da (20) | 8da (80) |
| 5 | 6c | 0.1 | 15 | min | 7ca (—) | 8ca (99) |

^{a}NMR yields.

Reactions of α,β-unsaturated aldehyde 6 and 2a were investigated and interesting results were shown as Eq. 2 and Table 2. For example, when crotonaldehyde 6a was used to react with 2a in the presence of 0.1 equiv of CAN at room temperature for 1 h, only 99% of 7aa was generated but no bis(indolyl)methane 8aa was observed when the mixture was checked by NMR or GCMS (entry 1). However, not only 64% of 7ba but also 29% of 8ba was also generated when b-methylcrotonaldehyde 6b was used (entry 2). Compared to 6a or 6b, the results of the use of 6c or 6d were slightly different and only 32% of 7ca and 66% of 8ca or 20% of 7da and 80% of 8da were generated (entries 3 and 4). We were surprised to find that only 99% of 8ea was generated when 6e was used (entry 5).

Based on Tables 1 and 2, we can conclude that the use of α,β-unsaturated ketone 1 can generate 4 and/or 5 and the use of α,β-unsaturated aldehyde 6 yield 7 and/or 8. The reaction mechanisms for the generation of 7 and 5 are all proposed to proceed through the 1,4-addition first and then to undergo the 1,2-addition but 8 is proposed to proceed through the 1,2-addition only which is different from the generation of 4 by proceeding through the 1,4-addition only. These different results possibly could be explained by the different steric hindrances between aldehyde and ketone. Aldehyde is always more reactive than ketone because the formyl group is much smaller than the acyl group. This assumption could also be proved by the fact that only 0.1 equiv of CAN is required for the aldehyde but at least 0.3 equiv of CAN is required for ketone under similar condition. In addition to the above description, the generation of the different products 7 and 8 from aldehydes such as 7aa from 6a and 8ea from 6e could also be explained by the presence of the different steric effects which were generated from the presence of the different groups at α and/or β carbon in these two substrates.

### EXAMPLE 3

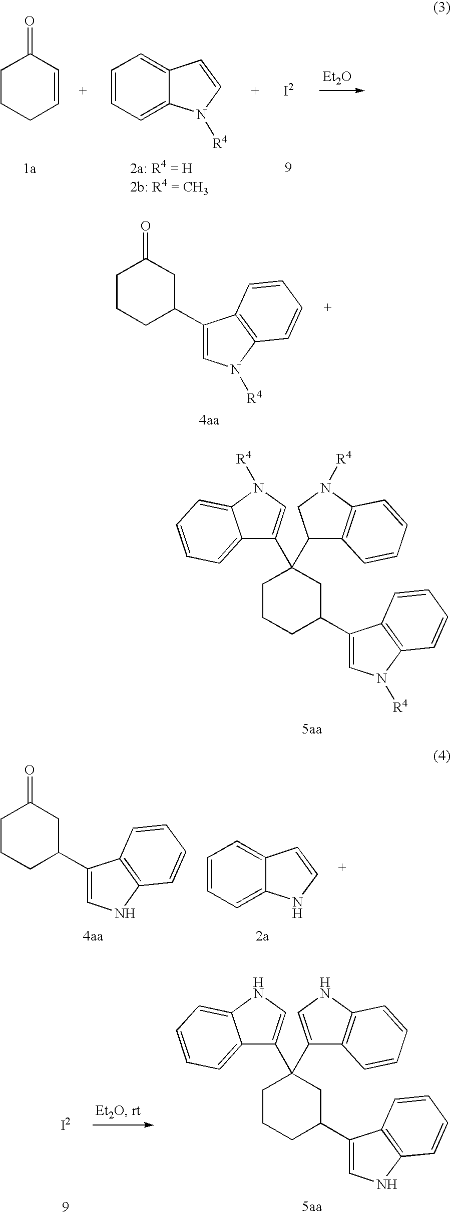

TABLE 3
Reaction ofα, β-unsaturated ketone 1 and indole 2a/2b in the
presence of molecular iodine

| | 1 (1 | 2 (4 | | | | |
|---|---|---|---|---|---|---|
| Entry | equiv) | equiv) | 1₂ (equiv) | Time (h) | 4^{a} (%) | 5^{a} (%) |
| 1 | 1a | 2a | 0.15 | 1.5 | 4aa (28) | 5aa (49) |
| 2 | 1a | 2a | 0.3 | 1 | 4aa (5) | 5aa (68) |
| 3 | 1a | 2a | 0.3 | 2 | 4aa (—) | 5aa (93) |
| 4 | 1a | 2a | 0.5 | 1 | 4aa (—) | 5aa (99) |
| 5 | 1a | 2a | 1 | 1 | 4aa (—) | 5aa (62) |
| 6 | 1a | 2b | 0.3 | 5/12 | 4ab (—) | 5ab (99) |
| 7 | 1b | 2a | 0.3 | ⅔ | 4ba (92) | 5ba (—) |
| 8 | 1c | 2a | 0.3 | ⅙ | 4ca (91) | 5ca (—) |
| 9 | 1d | 2a | 0.3 | 72 | 4da (43) | 5da (—) |
| 10 | 1e | 2a | 0.3 | 5 | 4ea (79) | 5ea (—) |

^{a}NMR yields.

We then changed CAN to iodine as a catalyst similar to the process of example 1, both 4aa and 5aa or only 5aa were observed when 1 equiv of 1a reacted with 4 equiv of 2a in the presence of different amounts of 12 in 1 mL of diethyl ether (Et₂O) solution (Eq. 3 and Table 3). When only 0.15 equiv of 12 was used, not only 4aa but also 5aa was isolated (entry 1). The most significant and interesting results were that only 93% or 99% of 5aa was obtained when 0.3 or 0.5 equiv of 9 I₂ (iodine) was used for 2 or 1 h under similar conditions (entries 3 and 4). Unfortunately, only 62% of 5aa was observed when 12 was increased to 1 equiv (entry 5). These results indicate that the use of 0.3 equiv of I₂ is good enough for this reaction. Based on the above condition, similar reactions were conducted by using 1a-d and 2a or 2b to obtain different yields of 4 and the results were shown as entries of 6-10. Compared to the results of Table 1 by using CAN, most of the substrates except 1d could produce moderate to high yields of 4 when the reaction was conducted in the presence of I₂. To substrate 1d, only 49% of 4da was generated compared to the use of CAN whose yield was 62%. The only difference is that all reactions were conducted in DMSO-H₂O solution by using CAN and in ether solution by using 12.

About the generation of 4aa and 5aa, the mechanism was proposed to proceed through the 1,4-addition first to obtain 4aa and then 4aa can react with 2a continuously to undergo the 1,2-addition to generate 5aa. In order to prove this assumption, 1 equiv of 4aa was used to react with 3 equiv of 2a in the presence of 0.3 equiv of iodine in 1 mL of ether for 1 h and 87% 5aa was isolated (Eq. 4). This result is good enough to explain why both 4 and 5 were generated when the same reaction was quenched or workup for shorter reaction time and only 5 was generated for longer reaction time.

### EXAMPLE 4

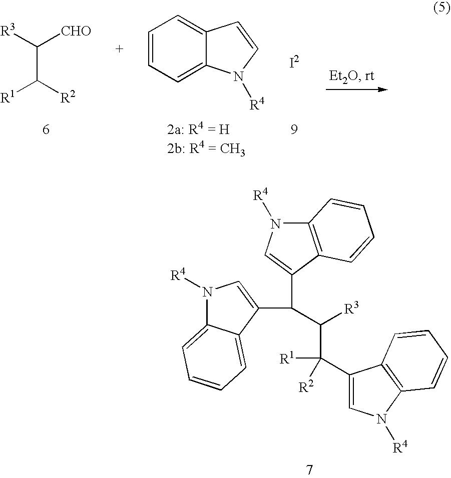

TABLE 4
Reaction of α, β-unsaturated aldehyde 6 and
indole 2 in the presence of molecular iodine

| Entry | 6 | 2 | I₂ (equiv) | Time | 7^{a} (%) |
|---|---|---|---|---|---|
| 1 | 6a | 2a | 0.1 | 15 min | 7aa (95) |
| 2 | 6a | 2b | 0.1 | 10 min | 7ab (86) |
| 3 | 6b | 2a | 0.1 | 10 min | 7ba (99) |
| 4 | 6b | 2b | 0.1 | 10 min | 7bb (76) |
| 5 | 6c | 2a | 0.1 | 3 h | 7ca (93) |
| 6 | 6c | 2b | 0.1 | 30 min | 7cb (95) |
| 7 | 6d | 2a | 0.1 | 5 min | 7da (28)^{b} |
| 8 | 6c | 2a | 0.1 | 10 min | 7ea (—)^{c} |
| 9 | R¹ = p-MeOC₆H₄, | 2a | 0.1 | 10 min | 7fa (79) |
| | R₂ = H, R₃ = H 6f | | | | |
| 10 | | 2b | 0.1 | 10 min | 7fb (82) |
| 11 | R¹ = o-MeOC₆H₄, | 2a | 0.1 | 10 min | 7ga (87) |
| | R₂ = H, R₃ = H 6g | | | | |
| 12 | | 2b | 0.1 | 10 min | 7gb (95) |

^{a}NMR yield.
^{b}46% of bis(indolyl)methane 8da was also generated.
^{c}99% of bis(indolyl)methane 8ea was generated.

Based on examples 1-3, similar reactions of 6 and 2 in the presence 0.1 equiv of 12 were also studied and the results were shown as Eq. 5 and Table 4. Compared to the results of Table 2, slight results were observed. To substrates 6a-c and 6f-g, only products 7aa-ca and 7fa-ga were generated but no products 8 were observed. Possible explanation for the different results may be assumed due to that CAN belongs to hard acid but 12 belongs to soft acid under similar reactions, so that most of the starting material 6a-c and 6f-g can undergo the 1,4-addition and then 1,2-addition predominately in the presence of 12. However, to 6d, 8da is the major products and to 6e, 8ea is the only product and both products were all proposed to be generated from the 1,2-addition only and these results were also similar to the results of the use of CAN. These special results can also be explained by the steric hinderance which was generated from the presence of different groups at β-carbon of aldehyde. This is the reason why these two substrates prefer to proceed thorough 1,2-addition to undergo the 1,4-addition and then 1,2-addition.

### EXAMPLE 5

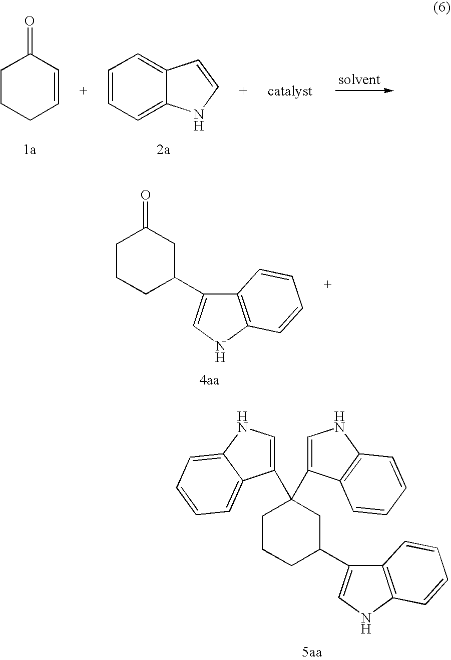

TABLE 5
Reaction of α, β-unsaturated ketone 1a with indole 2a catalyzed by
other catalysts

| | | | yield % |
|---|---|---|---|
| catalyst(mmole) | Time | solvent | (4aa/5aa) |
| InCl₃(0.1) | 1 day | EtOH | 23/4(S.M. 83) |
| CeCl₃(0.1) | 1 day | EtOH | 12/2(S.M. 72) |
| AlCl₃(0.1) | 1 day | EtOH | 10/90 |
| Dodecylbenzene sulfonic | 1 day | acetone/H₂O | 0/99 |
| Acid(0.1) | | | |
| 4-methylbenzenesulfonic | 1 day | Et₂O | 0/85 |
| acid(0.1) | | | |
| Amberlyst-15(0.1 g) | 1 day | EtOH | 19/11(S.M. 26) |
| TCT(0.05) | 1 day | Ether | 5/91 |
| NBS(0.1) | 2 days | EtOH | 7/24(S.M. 72) |

According to the results of examples 1-4, other catalysts are used to catalyze the reaction of starting compounds 1a and 2a and the result is shown in Eq. 6 and Table 5. As 0.1 equiv of metal halide is used as the catalyst, the yield of 4aa is higher in the case of indium halide (InCl₃) and cerium halide (CeCl₃) while the yield of 5aa is higher in the case of aluminum halide (AlCl₃) and the yield is as high as 95% in the entries 3 of Table 5. The yield of single product 5aa (experiment 4) is 99% by 0.1 equiv of dodecylbenzene sulfonic acid while the yield of single product 5aa (experiment 4) is 85% by 0.1 equiv of 4-methylbenzenesulfonic acid (experiment 5). On the contrary, the yield of the product 4aa is only 19% and that of the product 5aa is 11% by 0.1 g of the acidic ion exchange resin (experiment 6). In addition, the catalyst 2,4,6-trichloro-1,3,5-triazine (TCT) has excellent effect, producing 5% of the product 4aa and 91% of the product 5aa (experiment 7). For the catalyst N-bromosuccinimide (NBS), 7% of the product 4aa and 24% of the product 5aa (experiment 8) are produced.

### EXAMPLE 6

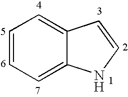

TABLE 6
Reaction of of α, β-unsaturated ketone 1 with indole and its
derivatives catalyzed by 0.3 equiv of iodine

| Indole and its | | | |
|---|---|---|---|
| deritives | Time (h) | solvent | yield % |
| 2a: 1-H(0.3) | 2 | Et₂O | 4aa/5aa = 0/93 |
| 2b: 1-CH₃(0.3) | 5/12 | Et₂O | 4ab/5ab = 0/99 |
| 2c: 5-F(0.3) | 2 | Et₂O | 4ac/5ac = 0/93 |
| 2d: 5-Br(0.3) | 2 | Et₂O | 4ad/5ad = 0/98 |
| 2e: 7-Et(0.15) | 1 | Et₂O | 4ae/5ae = 0/37 |

equiv of α,β-unsaturated ketone 1 and 4 equiv of indole and its derivatives 2a-2e are dissolved in 1 ml of diethyl ether (Et₂O) to form a solution. 0.15 or 0.3 equiv of iodine is used as the catalyst for the solution. Only one product 5 is produced and the result is shown in Table 6.

In the embodiment, the reason of producing different products may be due to steric effect of the starting compounds or reactants and/or acidity of the catalyst. The catalyst used in the invention has the advantages of high reaction efficiency, ease in handling reacted mixture, and mild or proper reaction conditions, such as carrying out the reaction under room temperature. In addition, the catalyst is low cost, easy to obtain, and low environmental impact and it satisfies current environmental protection trend.

A cell has the characteristics of proliferation, differentiation, and apoptosis. Coordination and regulation among proliferation, differentiation, and apoptosis of cells maintain a balanced-growing process for normal tissues. Especially, apoptosis plays an important role in cell death, renewal, and maintaining constant cell quantity. Many researchers have been mainly focused on proliferation activity of a tumor for a long time. Because of the limitations in experimental methods and means, the research on apoptosis disorder is very little and limited. However, there are more evidences showing that the apoptosis disorder is closely related to tumor formation. The tumor is not only a disease about the proliferation and differentiation disorders but also a disease about the apoptosis disorder. Recently, the apoptosis research has been drawn a great attention in life science.

As described in the prior art, apoptosis is also called “programmed cell death”. As described by Kerr, Wyllie, and Currie, apoptosis maintains stable cell environment and organizes cell death controlled by genes. In contrast to necrosis, apoptosis is not a passive process but an active process. Apoptosis is related to a series of gene initiation, expression, and regulation and is not a self-damaged phenomenon under pathological condition. Apoptosis is an initiative death process in order to adapt the survival environment better. Nuclear changes in apoptosis is that DNA of the cell chromosome cleavaged by endogenous endonuclease is fragmented between nucleosomes to produce a chromosome DNA fragment of multiples of 180-200 bp, that is chromosome DNA fragmentation. When apoptosis occurs in a cell, the cell membrane becomes shrinkage and dented, the chromatin becomes condensed and finally fragmented. Then, the cell membrane divides and surrounds the cytoplasm and also surrounds the DNA fragments of the cytoplasm to form a plurality of vesicular bodies with complete membrane structure, called apoptotic body. In the process of cell apoptosis, chromatin is condensed and cytoskeleton protein is destroyed by protease. But, the main cell organelle, such as mitochondrion and lysosome, maintains its structure and function until the late stage of apoptosis. Endoplasmic reticulum still has the function of synthesizing proteins during early stage of apoptosis, and then expands to become bubble and thereby to be contacted and fused with cell membrane to form cytoplasmic bubble. The cell membrane always remains as a whole and thus content is not spilled. Therefore, no inflammation occurs.

Therefore, apoptosis is a very special and natural cell process controlled by many genes, such as pro-apoptotic gene: p53, Bax, Bad, Bak, and anti-apoptotic gene: Bcl-2, Bcl-xL, Bcl-w, etc. Cells are undergoing self preset procedures until cells are swallowed in order to maintain homeostasis cells and tissues. There are four extrinsic characteristics in apoptosis: (1) shrinkage of cytoplasm; (2) condensation of chromosome; (3) DNA fragmentation; and (4) production of apoptotic bodies. Its characteristic is that cell membranes do not crack and content are not spilled. Thus, no inflammation occurs. In the process of cell apoptosis, double strand DNA in the cell is cleavaged by caspase to form a size of about 300 bp and further to be fragmented to about 185 bp nucleosome. Finally, apoptotic body is formed and then swallowed and removed by phagocyte. Apoptosis has many important functions on animal growth and development, such as morphological change, removal of unnecessary structures, control of cell quantity, removal of abnormal, malfunctioned, harmful cells, and production of differentiated cells, etc. In the experimental method of detecting cell death comprises: (1) electrophoresis separation technique: extracting DNA of the apoptotic cell and utilizing electrophoresis separation technique to observe DNA ladder assay to find out the degree of DNA fragmentation; and (2) flow cytometry:analyzing the ratio of each cell cycle where the flow cytometer is at a normal cell cycle, comprising: G1 (Gap 1), S (synthesis), G2 (Gap 2), and M (mitosis) cycles, if cells die, and cells may be apoptosis if cells at sub-G1 cycle are detected.

In a third embodiment of the present invention, a medical composition for cancer treatment is disclosed. The medical composition comprises a compound with the following general structure:

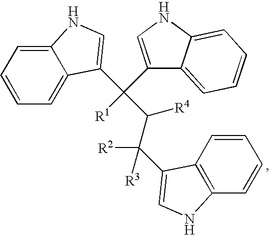

its enantiomers, diastereomers or pharmaceutically acceptable salts thereof or any combination of the above, wherein R¹, R², R³, and R⁴ are selected from the group consisting of the following: hydrogen atom, alkyl group, substituted alkyl group, aryl group, substituted aryl group; or any two of the R¹, R², R³, and R⁴ form cyclic group.

In a preferable example of this embodiment, the compound comprises one selected from the following group:

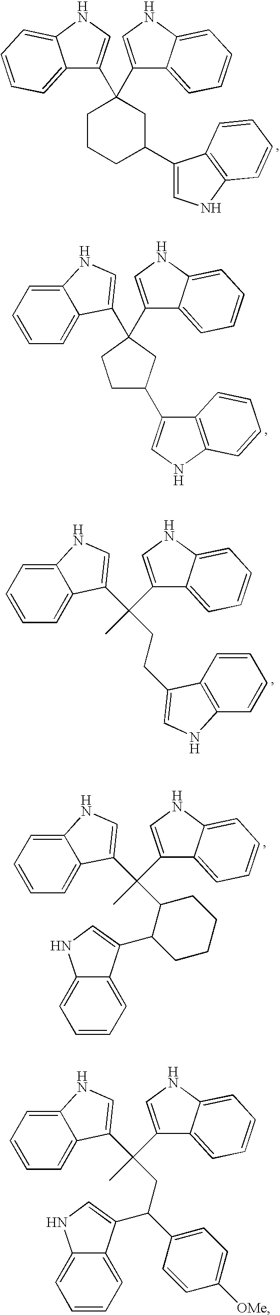

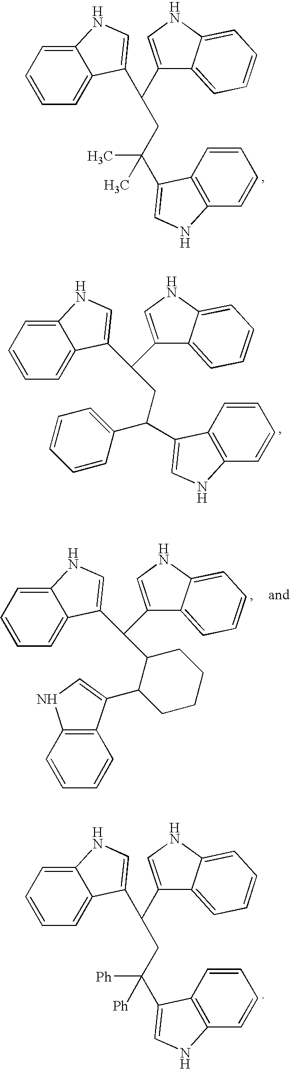

In this embodiment, the cancer is one selected from the following group or any combination: lung cancer, esophageal cancer, ovarian cancer, breast cancer, lymphoma cancer, pancreatic cancer, colorectal cancer, head and neck cancer, and bladder cancer. The 3-indole based compound is especially suitable for the cancer cells with mutation of the tumor suppressor gene p53, such as lung cancer cells. Clinically, lung cancers can be divided into two categories, small cell lung cancers (SCLCs) and non-small cell lung cancers (NSCLCs). Most patients belong to non-small cell lung cancers. The non-small cell lung cancers comprise adenocarcinoma lung cancers and squamous cell lung cancers, and large cell lung cancer. Among these, adenocarcinoma lung cancers are often seen in lung cancer patients. Generally, non-smokes belong to adenocarcinoma lung cancers while smokers belong to squamous cell lung cancers.

### EXAMPLE 7

### Material and Method

### Cell Lines

Five lung cancer cell lines are used in the experiment. Their p53 gene types are described as the following: H1299 (p53 null type), CL1-1 (p53 mutant type), H1435 (p53 mutant), H1437 (p53 mutant), and A549 (p53 wild-type). In addition, the normal lung cell line IMR⁹⁰ is used as the control group. Two esophageal cancer cell lines are KYSE 170 and KYSE510. H1299, CL1-1, H1437, A549, and IMR⁹⁰ are cultured in a Dulbecco's modified Eagle's medium (DMEM) nutrient fluid containing 10% of fetal bovine serum, while H1435, KYSE170, and KYSE510 are cultured in a RPMI-1640 nutrient fluid containing 10% of fetal bovine serum.

### Drug Treatment

The solvent for medicine is dimethyl sulphoxide (DMSO). Cells are cultured in a humidified incubator at 37° C. and 5% of CO₂ with an appropriate culture medium. A fixed amount (about 3×10⁵) of cells are cultured for 12˜16 hrs in a six-well culture dish. On the next day, the cells are cultured in an appropriate culture medium with different concentrations of the 3-indole anticancer agents at 37° C. for 24 hrs. The survival rate of the cells after drug treatment is counted to find out the required concentration of the 3-indole anticancer agents (i.e., IC50) while the tumor cell death rates reaches 50% compared to untreated cells.

### Cell Survival Test (MTT Assay)

Cells are cultured in a humidified incubator at 37° C. and 5% of CO₂ with an appropriate culture medium. A fixed amount (about 3×10⁵) of cells are cultured for 12˜16 hrs in a six-well culture dish. On the next day, the cells are cultured in an appropriate culture medium with different concentrations of the 3-indole anticancer agents at 37° C. for 24 hrs and then the culture medium is removed. After, an appropriate culture medium with MTT [3-(4,5-cimethylthiazol-2-yl)-2,5-dphenyl tetrazolium bromide] 1 ml/well culture medium is added. It is placed in the humidified incubator for 1 hr. The culture medium containing MTT is re removed and 600 μl/well of DMSO is added. It is then placed in a horizontal shaker under the environment avoiding light for 5 minutes. 200 μl/well is taken and then placed in a 96-well culture dish for testing absorbance at 570 nm. The absorbance is then converted to the number of cells to obtain the cell survival rate.

### DNA Fragmentation Analysis (DNA Ladder Assay)

Culture cells are scraped from the culture dish and then is added with 20 μl of protease K and 200 μl of AL buffer. The mixture is blended for 15 seconds to break up the cells and placed on a dry bath incubator at 56° C. for 10 seconds. 99% ethanol 200 μl is added. The mixture solution is moved to the column of QIAGEN kit to centrifugal separation 8000 rpm for 1 minute and then the lower-layered solution is removed. Add 500 μl of AW1 buffer of QIAGEN kit to centrifugal separation 14000 rpm for 3 minute, and then the lower-layered solution is removed. Add 500 μl of AW2 buffer of QIAGEN kit to centrifugal separation 14000 rpm for 3 minute. The upper layer column is moved to a new 1.5 ml eppendorf, 200 μl of AE buffer is added at room temperature for 1 min, and then centrifugal separation 8000 rpm for 1 min is carried out. Finally, electrophoresis is carried out for the collected DNA to observe DNA ladder assay.

### Cell Cycle Assay

A fix amount (about 2×10⁶) of cells are fixed by 70% ethanol and stood still at −20° C. in a refrigerator for 24 hrs. Low speed centrifugal separation with 900 rpm for 5 minutes is carried out and the upper clear solution is sucked and removed. Cell lump is evenly dispersed and 5 ml phosphate-based saline (PBS) is added to clean cells for three times. Staining solution of 1.0 ml of propidium iodide (PI)/Triton X-100 is added. Cell lump is evenly dispersed and shaken to be mixed and reacted at room temperature in a dark room for 30 minutes where the final concentration of Triton-X is 0.1%, that of RNase A is 0.2 mg/ml, and that of PI is 20 μg/ml. The sample is then mixed and filtered with a 35 μm nylon screen. The fluorescence intensity of PI in the cells detected by FACScan flow cytometry (BD, Mountain View, Calif.) is used to estimate DNA distribution in each cell cycle for about 10,000 cells and thereby to define each stage of the cell cycle. Mofit LT Ver 2.0 software is used to calculate the ratio of each cell cycle distribution. If the cell cycle is affected by the drug, the cell cycle distribution of the treated sample detected by the instrument will be different from that of the untreated sample. The cell cycle distribution includes G1, S, G2, and M phase. G1 represents “Gap 1”. S represents “DNA synthesis”, i.e. DNA replication. M represents “mitosis”, performing nuclear division (disjunction of chromosome) and cytokinesis. If a cell is detected in the phase of sub-G1, the cell may have DNA fragmentation appeared in apoptosis.

### Western Bolt

Culture cells are added into optimum amount of RIPA [50 mM Tris pH8.0, 150 mM NaCl, 0.5% sodium deoxycholate, 0.1% SDS, 1% Triton X-100, 5 mM phenylmethylsulphonyl fluoride (PMSF), 10 mg/ml leupeptin, 20 mM sodium phosphate pH7.0]. After dissolved, cells are scraped by a cell scrapper and treated by a centrifuge with 10,000 rpm at 4° C. for 30 minutes. Then, 4 μl of the upper liquid is taken to do protein quantitative analysis. The rest of liquid are packaged and stored at −20° C. Protein quantitative method used Bio-Rad DC (Detergent-Compatible) Protein Assay, where 5 μl standard or sample solution is added into a 96-well culture dish, 25 μl of alkaline copper tartrate solution is added, then 200 μl of Folin reagent is added, the mixture is mixed and reacted for 15 mins, and then the absorbance at 630 nm is measured. 0.5 [symbol] 1.0 [symbol] 1.5 [symbol] 2.0 mg/ml of bovine serum albumin standard are used as the standard albumin to obtain a calibration curve. The calibrated curve is then fitted with linear regression to obtain the regression line for calculating the concentration (mg/ml) of the proteins in the sample.

Electroblotting: 30 μg of proteins is added to 3× sample buffer solution, comprising 350 mM Tris-HCl pH 6.8, 12% SDS, 0.02% bromophenol blue, 35% glycerol, and 30% mercaptoethanol. After the solution is boiled for 5 mins. Then, electrophoresis is carried out for the collected proteins. Electrophoresis for the sample is carried out at 80 Volts of voltage is applied for 10mins and then at the 130V for 70 mins.

Immunosorbent assay: The proteins on SDS-PAGE blot onto a PVDF membrane. The membrane then soaks in the blocking solution (10% skim milk in PBS-T) to react at room temperature for 1 hr. It is then washed by PBS-T (phosphate-based saline-0.5% Tween-20) twice in which it takes 10 mins each time. Primary antibody Bcl-2 (Cell Signal Technology, Beverly, Mass. 01915, USA) and glyceraldehyde 3-phosphate dehydrogenase (GAPDH) (Novus Biologicals, Littleton, USA) are then added to react at room temperature for 2 hrs. It is then washed by PBS-T three times in which it takes 10 mins each time. Secondary anibody HRP-goat antirabbit IgG is added to react at room temperature for 1 hr. It is then again washed by PBS-T three times in which it takes 10 mins each time. Finally, ECL Chemiluminescent Western System (Amersham, Arlington Height, Ill., USA) reagent is added on the membrane to react for 5 mins under the environment avoiding light in older to expression reaction signal.

### In Vivo Antitumor Activity Test

5×10⁶ of A549 cancer cells are washed by one time with PBS and then desolved by 100 μl HBSS (Hanks' Balanced Salt Solution). HBSS cell suspension solution is injected into the subcutaneous tissue of the back of nude mice (ICR-Foxn 1 nude mice, national laboratory animal center). After injected with tumor cells for about 10-14 days and when the tumor grows up to a size of 50 mm³, the new 3-indole based anticancer agent or the traditional chemotherapy medicine Taxol and solvent is injected to the intraperitoneal of nude mice. It is injected once every other day (day 0, 2, 4, 6, 8) and one dose is 0.2 mg. The total injection is five times and the total dosage is 50 mg/Kg. For a period of 30 days, (1) the tumor size and change are observed and recorded where the longest side a and the shortest side b of the tumor are used to define the volume of the tumor as (a×b²)/2; (2) At the end of the experiments, animals were euthanized with carbon dioxide inhalation, followed by cervical dislocation. In the meantime the blood of the mice after experiments is used to have serum biochemistry toxicities test; and (3) the liver and kidney sections of the mice are used to be histopathologic observed.

### EXAMPLE 8

### Result

### Cell Toxicity Test

The experiment of the cytotoxicity of indole-based compounds, 3-indole, for various tumor cells shows that the 3-indole based compound is very efficacious against lung cancer lines with various status of p53: H1299 (p53 null type), CL1-1 (p53 mutant type), H1435 (p53 mutant), H1437 (p53 mutant), and A549 (p53 wild-type); and two esophageal cancer cell line: KYSE170 and KYSE510 and suppressing the growth of cancer cells can be achieved with a low dosage. In addition, for normal lung cells IMR90, there is no obvious apparent cyclotoxicity while treated with the same dosage. Therefore, indole-based compounds show potential to become a novel anticancer drug.

### Animal Test

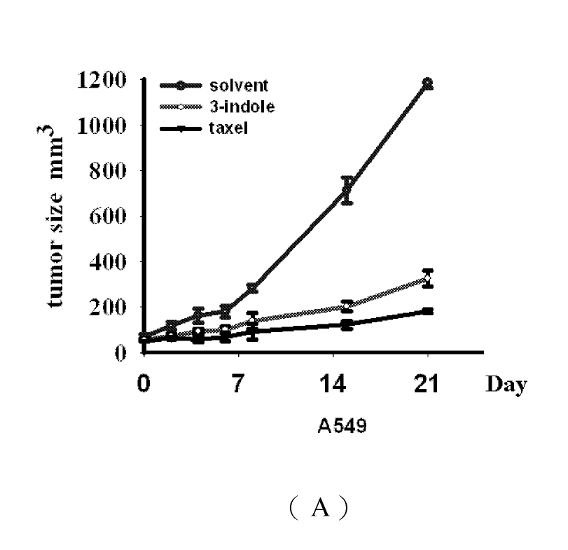

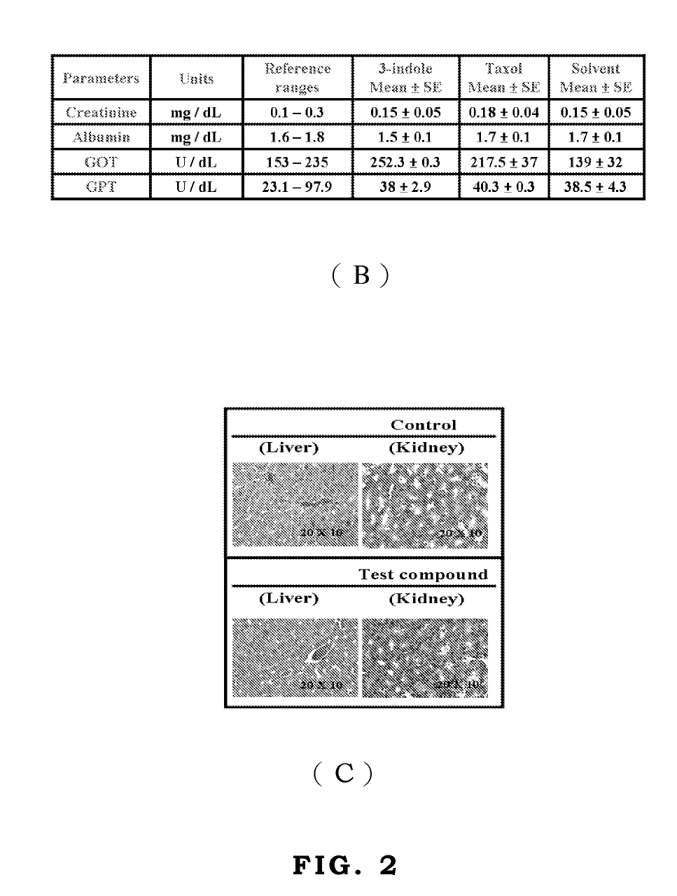

In the in vivo antitumor experiment and drug metabolism identification analysis, the A549 lung cancer cells are injected to the subcutaneous tissue of the back of nude mice (ICR-Foxnl). After, when the tumor grows up to a size of 50 mm³, 3-indole based compound is injected intraperitoneal of nude mice. The traditional chemotherapy medicine Taxol is the positive control group and the solvent for 3-indole based compound, DMSO, is the negative control group. Referring to FIG. 2(A), compared to the control group, after 3-indole based compound is injected, it is assured that the growth of the tumor formed by the lung cancer cells A549 is suppressed up to 30˜50%. On the other hand, as shown in FIG. 2(B), in analysis of the blood and biochemical test, after 3-indole based compound is injected, the blood biochemical values are all within the normal range [glutamic oxaiacetic transaminase (GOT) and glutamic pyvuvic transaminase (GPT)], determined by clinician and laboratory staff. The H&E staining result of the tissue section, after determined by the pathologist, it is assured that the 3-indole based compound injection is not harmful to the related organs of the mice, as shown in FIG. 2(C).

### Cell Cycle and Program Cell Death Identification

In order to understand the mechanism of suppressing the tumor cell growth by 3-indole based compound, the experiments by flow cytometry, DNA ladder assay, and Western bolt, are carried out to observe the cell distribution of each stage in the cell cycle and to identify program cell death. Referring to FIG. 3, by the experiment of flow cytometry, 3-indole based compound has the effect on the cell cycle with different dosages. After the lung cancer cell (A549, H1299, H1437, H1435, CL1-1) is treated by 10 μM dosage for 24 hrs, the cell cycle remains in G1. While it is treated by 30 μM dosage, the cells remaining in sub-G1 are further increased, that shows the phenomenon of program cell death. Referring to FIG. 4, the experiment of DNA ladder assay shows that the death mechanism of the lung cancer cell (A549, H1299, H1437, H1435, CL1-1) goes through cell apoptosis and thus DNA is regularly fragmented. There are several known apoptosis-related molecules, such as Bcl-2 (B-cell leukemia/lymphoma), involved in carrying out cell apoptosis continuous reaction. Referring to FIG. 5, by Western blot analysis, it is assured that 3-indole based compound induced apoptosis is related to expression of Bcl-2 family.

The invention discloses a new compounds, 3-indole based compound, through phytochemical indole structure, that can induce apoptosis. 3-indole based compound is very efficacious against to p53 wild-type A549 cells, p53 mutant H1437, H1435, and CL1-1 cells, p53 null type H1299 cell and can suppress the growth of the tumor in the animal experiment. Furthermore, in vitro cellular singling transduction pathway analysis shows that 3-indole drug induces apoptosis through Bcl-2 family pathway and can suppress the growth of the cancer cell with the mutations of various p53 status. It shows that indole-based compounds, 3-indole, has potential to become a new anticancer drug to increase the cure rate for various types of cancer patients.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A method for forming indole-based compounds, comprising: a catalyzed reaction of α, β-unsaturated ketone and indole or its derivatives in the presence of at least one Lewis acid;
wherein the operation temperature of said catalyzed reaction is lower than or equal to 30° C.; said Lewis acid comprises one selected from the group consisting of the following: organic sulfonic acid (sulfonate), halogen, inorganic ammonium salt, and metal halide; and the general structure of said indole of its derivatives is as following: 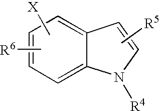
in which X is halogen and R⁴, R⁵, and R⁶ are independently selected from the group consisting of the following: hydrogen atom and linear-chained alkyl group.

2. The method according to claim 1, wherein said organic sulfonic acid comprises one selected from the group consisting of the following: alkyl sulfonic acid (sulfonate), aryl sulfonic acid (sulfonate), alkyl aryl sulfonic acid (sulfonate), sulfonated styrene-divinylbenzene copolymer, and Nafion.

3. The method according to claim 1, wherein said inorganic ammonium salt comprises cerium inorganic ammonium nitrate (CAN).

4. The method according to claim 1, wherein said metal halide comprises one selected from the group consisting of the following: indium halide (InCl₃), cerium halide (CeCl₃), and aluminum halide (AlCl₃).

5. The method according to claim 1, wherein said α, β-unsaturated ketone comprises one selected from the following group:

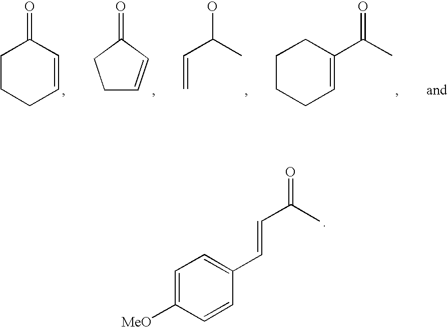

6. A method for forming indole-based compounds, comprising: a catalyzed reaction of α, β-unsaturated aldehyde and indole or its derivatives in the presence of at least one Lewis acid;
wherein the operation temperature of said catalyzed reaction is lower than or equal to 30° C.; said Lewis acid comprises one selected from the group consisting of the following: organic sulfonic acid (sulfonate), halogen, inorganic ammonium salt, and metal halide; and the general structure of said indole of its derivatives is as following: 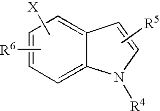
in which X is halogen and R⁴, R⁵, and R⁶ are independently selected from the group consisting of the following: hydrogen atom and linear-chained alkyl group.

7. The method according to claim 6, wherein said organic sulfonic acid comprises one selected from the group consisting of the following: alkyl sulfonic acid (sulfonate), aryl sulfonic acid (sulfonate), alkyl aryl sulfonic acid (sulfonate), sulfonated styrene-divinylbenzene copolymer, and Nafion.

8. The method according to claim 6, wherein said inorganic ammonium salt comprises cerium inorganic ammonium nitrate (CAN).

9. The method according to claim 6, wherein said metal halide comprises one selected from the group consisting of the following: indium halide (InCl₃), cerium halide (CeCl₃), and aluminum halide (AlCl₃).

10. The method according to claim 6, wherein said α, β-unsaturated aldehyde comprises one selected from the following group:

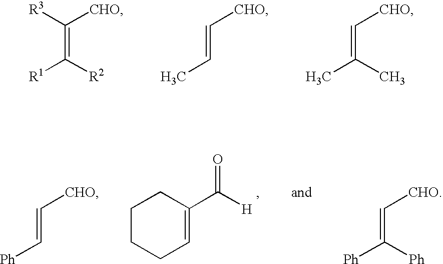

11. A medical composition for cancer treatment, comprising a compound with the following general structure: 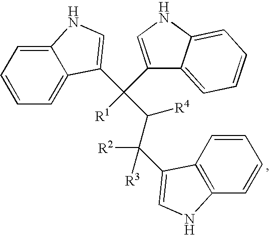
its enantiomers, diastereomers or pharmaceutically acceptable salts thereof or any combination of the above, wherein R¹, R², R³, and R⁴ are selected from the group consisting of the following: hydrogen atom, alkyl group, substituted alkyl group, aryl group, substituted aryl group; or any two of the R¹, R², R³, and R⁴ form cyclic group.

12. The medical composition according to claim 11, wherein said compound comprises one selected from the following group:

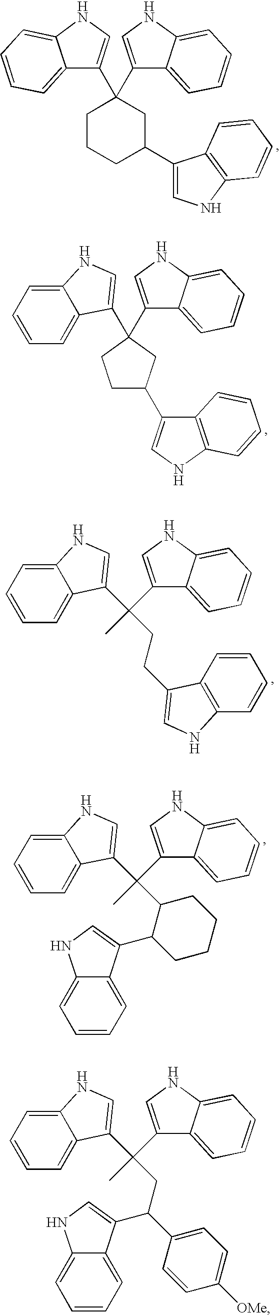

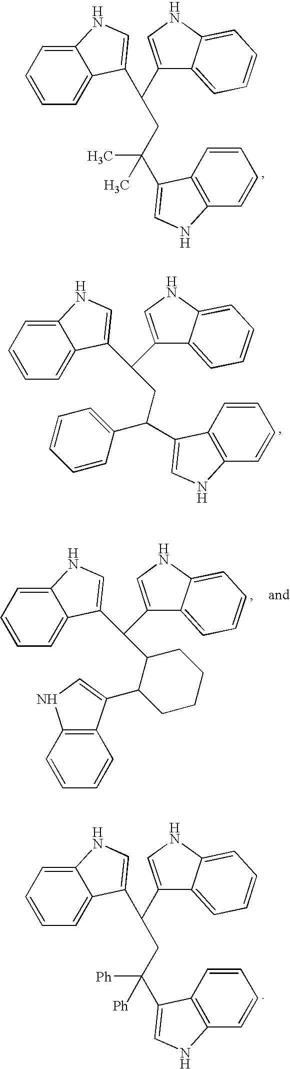

13. The medical composition according to claim 11, wherein the tumor suppressor gene p53 in the cancer cells various mutates.

14. The medical composition according to claim 11, wherein said cancer is one selected from the following group or any combination: lung cancer, esophageal cancer, ovarian cancer, breast cancer, lymphoma cancer, pancreatic cancer, colorectal cancer, head and neck cancer, and bladder cancer.

15. The medical composition according to claim 11, wherein said cancer is non-small cell lung cancers.

16. The medical composition according to claim 11, wherein the death of the cancer cells is through apoptosis.

17. The medical composition according to claim 11, wherein the medical composition is used in suppressing cell division of the cancer cells.

18. A method for cancer treatment on mammals, which comprises administering to the mammals a therapeutically effective amount of the medical composition according to claim 11.
